(19) United States
(12) Patent Application Publication
(10) Pub. No.: US 2018/0236272 A1
(43) Pub. Date: Aug. 23, 2018

(54) TISSUE STABILIZATION FOR THERAPEUTIC ULTRASOUND

(71) Applicant: SONACARE MEDICAL, LLC, Charlotte, NC (US)

(72) Inventor: Mark Carol, Charolotte, NC (US)

(21) Appl. No.: 15/955,943

(22) Filed: Apr. 18, 2018

Related U.S. Application Data
(63) Continuation-in-part of application No. 14/938,995, filed on Nov. 12, 2015, now Pat. No. 9,974,983.

(51) Int. Cl. A61N 7/02

(52) U.S. Cl. CPC A61N 7/02; A61N 2007/0082; A61N 7/022

(57) ABSTRACT

A device including a focused ultrasound probe, a first balloon, and a second balloon. The first balloon can be located at least partially inside the second balloon. The inner balloon can be configured to act as a fluid interface, a mechanism for cooling, and a mechanism for changing tissue depth of a focal point of the probe. The second balloon can be filed with a thermosensitive hydrogel configured to turn from a fluid at room temperature to a gel at physiologic temperatures. The first and second balloons can be configured to create a fluid interface between the probe and targeted tissue of a patient.

### CROSS-REFERENCE TO RELATED APPLICATIONS

The present application is a continuation-in-part (CIP) application of U.S. patent application Ser. No. 14/938,995, titled “Tissue Stabilization for Therapeutic Ultrasound” and filed Nov. 12, 2015, the disclosure of which is hereby incorporated by reference.

### SUMMARY

Focused ultrasound can be used to deliver energy concentrated in targeted tissue on or inside a patient, while minimizing the amount of energy deposited in any other tissue between the probe delivering the energy and the targeted tissue. In order to deliver correctly the desired amount of energy to the targeted or localized tissue, the probe must be positioned and stabilized correctly geometrically relative to the targeted or desired tissue and held in that position during the course of treatment. However, there are operating principles that make this difficult to achieve.

Satisfactory delivery of therapeutic focused ultrasound benefits from a lack of air and bone, as well as other obstructions along the path, between the transducer and the target tissue. This necessitates usually the creation of a liquid coupling interface between the probe and tissue, with the liquid being contained within a membrane or balloon. The volume of liquid can be increased or decreased to adjust the position of the probe relative to the tissue—increasing the volume pushes the probe further from the surface tissue and brings the focal point of the probe closer to the surface, while decreasing it brings the probe closer to the surface tissue thereby pushing the focal point of the probe deeper into the tissue. It is desirable for the liquid to exist in a fluid state so it can be circulated and used to remove excess heat from the surface of the probe, thereby extending the life of the crystal(s) used to generate the focused ultrasound and ensuring proper performance. However, surrounding the probe with a “fluid filed sac” or fluid interface can introduce the potential for sac or interface to move relative to the target tissue and, therefore, the probe to move relative to the target tissue.

Therefore, it would be desirable to produce a means that will allow a fluid interface between the probe and the tissue to be created without destabilizing the interface between the probe and the tissue.

In one embodiment, the present disclosure includes a device and/or method configured to create a fluid interface between a focused ultrasound probe and tissue containing targeted tissue, while also stabilizing the geometric position between the probe and the tissue. A double balloon can be provided, with one distensible balloon located inside the other distensible balloon, the inner balloon being filled with water or another non-gaseous fluid, where the inner balloon acts as a fluid interface and a mechanism for cooling and a mechanism for changing the tissue depth of the focal point, the outer balloon being filled with a thermosensitive hydrogel that turns from a fluid at room temperature to a gel at physiologic temperatures, thereby allowing the thermosensitive hydrogel to act to immobilize the inner balloon relative to the outer balloon and the outer balloon relative to the surrounding tissue.

In a further embodiment, the present disclosure includes a method for treating tissue with focused ultrasound, the method can include: inserting a dual balloon port into a natural or man-made orifice in a patient; inserting a probe capable of delivering therapeutic ultrasound into a first, inner balloon; filling the inner balloon with a free flowing liquid or another non-gaseous fluid; positioning the balloon complex in the correct position for treating the targeted tissue; filling a second, outer balloon with enough or sufficient thermosensitive hydrogel to engulf or achieve significant contact with the region of tissue containing the targeted tissue; adding fluid to the inner balloon to create the proper delivery depth if required; allowing or forcing the hydrogel to undergo a sol-gel transition; delivering the ultrasound therapy; allowing or forcing the hydrogel to undergo a gel-sol transition; removing the hydrogel from the outer balloon; removing the free flowing fluid from the inner balloon; removing the probe from the inner balloon; removing the balloon port from the tissue; whereby an ultrasound treatment can be delivered with a stable geometry between the ultrasound probe and the targeted tissue and with correct and safe functioning of the ultrasound probe.

As is to be appreciated by one skilled in the art, one or more aspects of the foregoing disclosed systems and methods may be combined or even omitted, if desirable.

### DRAWINGS

The foregoing summary, as well as the following detailed description of the invention, will be better understood when read in conjunction with the appended drawings. For the purpose of illustrating the invention, there are shown in the drawings various illustrative embodiments. It should be understood, however, that the invention is not limited to the precise arrangements and instrumentalities shown. In the drawings:

FIG. 1 is a schematic representation of a system according to an embodiment of the present invention;

FIG. 2 is a schematic representation of at least a portion of a probe according to an embodiment of the present invention;

FIG. 3 is a schematic representation of a balloon complex of the system in a secondary or expanded state;

FIG. 4 is a schematic representation of a probe inserted into a cavity of a patient, wherein the balloon complex is in at least a partially collapsed or constricted state; and

FIG. 5 is a schematic representation of a probe inserted into a cavity of a patient, wherein the balloon complex is in at least a partially expanded state.

### DETAILED DESCRIPTION

Various embodiments of the present disclosure are described hereinafter with reference to the figures. It should be noted that the figures are not drawn to scale and elements of similar structures or functions are represented by like reference numerals throughout the figures. It should also be noted that the figures are not intended to facilitate the description of specific embodiments of the invention. The figures are not intended as an exhaustive description of the invention or as a limitation on the scope of the invention. In addition, an aspect described in conjunction with a particular embodiment of the present invention is not necessarily limited to that embodiment and can be practiced in any other embodiments of the present invention. It will be appreciated that while various embodiments of the invention are described in connection with focused ultrasound treatment of tumors, the claimed invention has application in other industries and to targets other than cancers. Unless specifically set forth herein, the terms “a,” “an” and “the” are not limited to one element, but instead should be read as “at least one.”

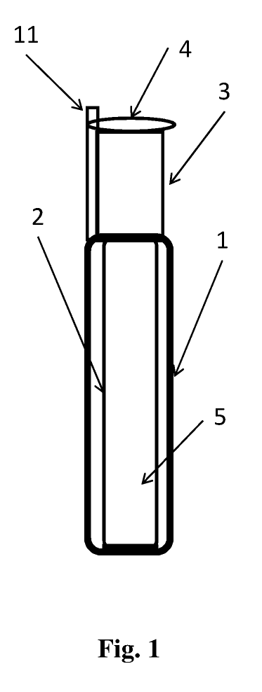

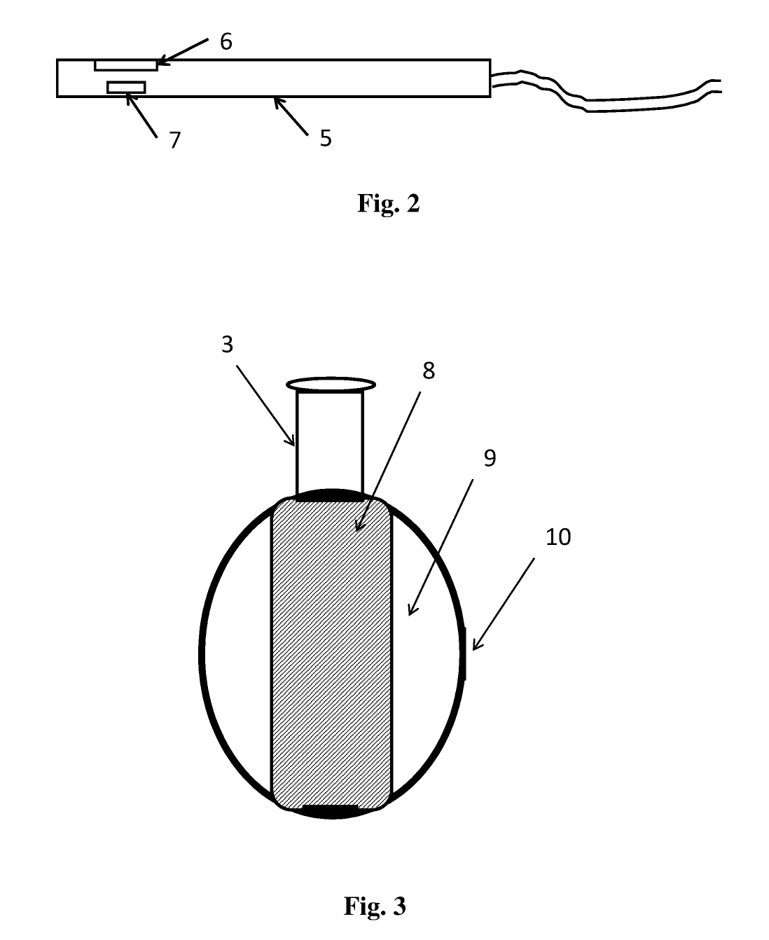

Referring to FIGS. 1-5, the present disclosure relates to a system for performing a surgical procedure. In one embodiment, the system can include at least one transducer positioned in or on a probe 5 (as understood by those skilled in the art) used to deliver thermal energy to a designated treatment volume 17 through one or more therapy crystals 6. The transducer may be configured to include one or more imaging crystals 7 used to generate one or more images of the designated treatment volume and surrounding tissue. In one embodiment, each therapy crystal 6 and imaging crystal 7 is separate or spaced-apart from a distal tip of the probe 5 (see FIG. 2). The images can be used to position correctly the volume of tissue targeted for the delivery of thermal energy. A user input interface can be provided to define the region, volume, and/or location of tissue designated to receive the thermal energy. The transducer can include one or more individual therapy crystals, each with a fixed focal point acting independently, or it can include one or more groupings of multiple crystals, which together form an annular, linear, 2-D, or other form of array where the focal point of each group of crystals can be moved electronically. The probe 5 can include one or more motors integral to its housing used to translate and/or rotate the transducer relative to the probe housing or can use one or more motors external to the probe 5 to translate and/or rotate the transducer relative to the probe housing.

Referring to FIG. 1, the system can include a balloon complex having a balloon-within-a-balloon design. More particularly, in one embodiment, the system can include a first, inner balloon 2 wholly contained within a second, outer balloon 1, except for at their necks 3 where each balloon 2, 1 can have its own ingress and egress port(s). The inner balloon 2 can be designed to be symmetrically or evenly expandable 8 (see FIG. 3) or can be constructed so that it expands at different rates, and to different degrees, in different directions. Thus, in one embodiment, the first balloon 2 is expandable to form an elliptical or circular shape having a convex exterior surface without any concave portions, which is distinct from the preselected concave shape of lens balloon 817 of U.S. Publication No. 2002/0068885. Regardless, in at least one embodiment, the inner balloon 2 will remain taut when expanded (see FIG. 3) and would not conform to a contacting surface. The outer balloon 1 can be designed to expand in a free-form manner 9 (see FIG. 3), depending, for example, on the tissue 18 against which it comes in contact, allowing it to adopt or conform (at least in part) to the shape of that tissue 18. Alternatively, the outer balloon 1 can be designed to be symmetrically or evenly expandable (similar to one embodiment of the inner balloon 2 shown in FIG. 3), which is distinct from the preselected concave shape of outer balloon 811 of U.S. Publication No. 2002/0068885. As a further alternatively, the outer balloon 1 can be constructed so that it expands at different rates, and to different degrees, in different directions, etc.

In one embodiment, a single port (for both ingress and egress of fluid) can be provided for each balloon 2, 1 at or near the neck 3 thereof. Each port can be either self-sealing or can be controlled with a valve, luer lock, or similar mechanism. Alternatively, an egress port and a separate ingress port can be provided for each balloon 2, 1 at or near the neck thereof. In such an embodiment, the egress port can be spaced-apart or separate and distinct from the ingress port. For the outer balloon 1, the port(s) can be used to remove air from the outer balloon 1 and to inject and remove a fluid, such as a hydrogel. For the inner balloon 2, the port(s) can be used to remove air from the inner balloon 2 and pass the ultrasound probe at least partially into the inner balloon 2. Water or another fluid (e.g., a non-gaseous fluid, such as liquid, gel or another non-gaseous fluid capable of creating an echogenic interface) can be injected into the inner balloon 2 through the port(s) or it can be instilled into the inner balloon 2 through an opening in the ultrasound probe 5. In one embodiment, the non-gaseous fluid injected into the inner balloon 2 flows around the transducer(s) to create an echogenic interface. In one embodiment, both the inner and outer balloons 2, 1 are completely filed with the water, liquid, and/or gel, which prevents any bubbles that may be present from interfering with the ultrasound propagation through the structure.

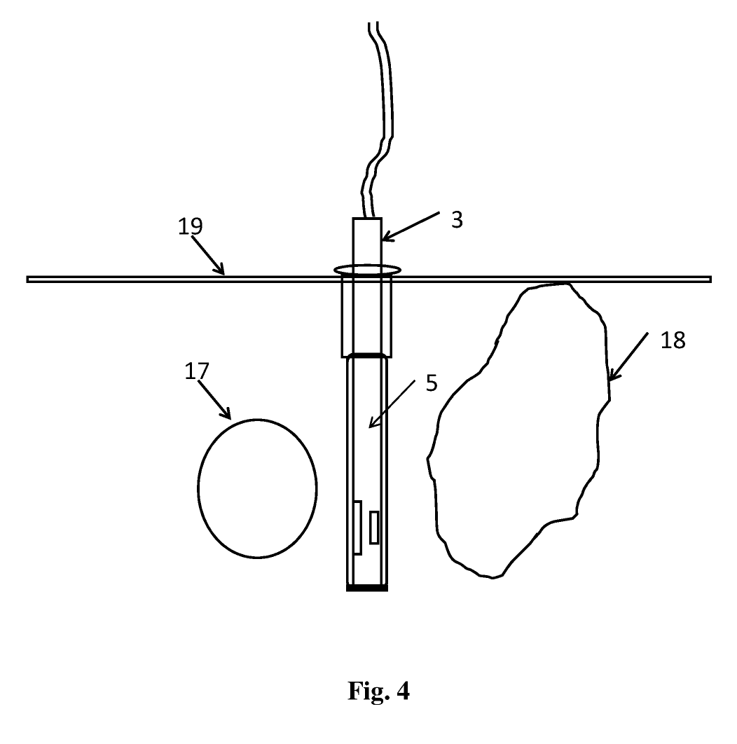

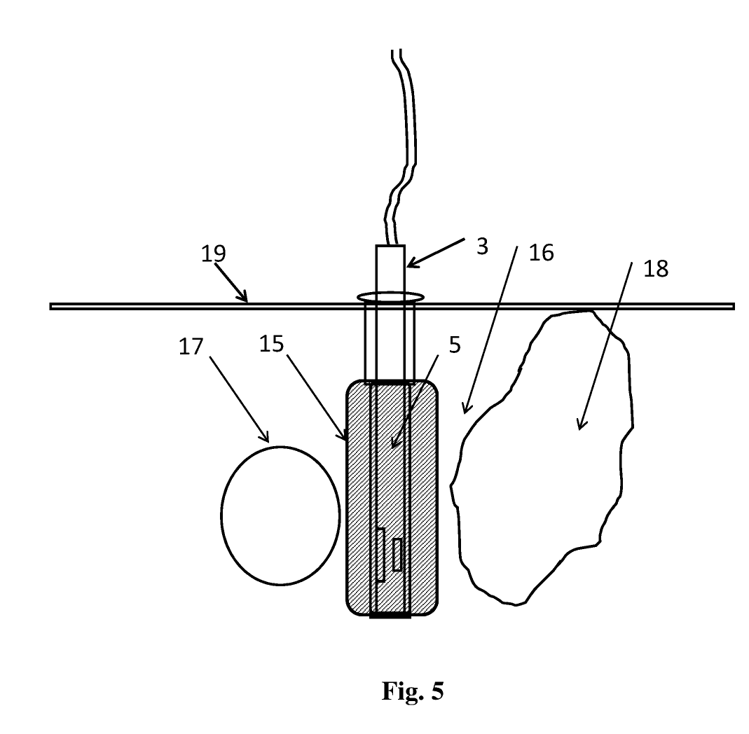

The neck 3 common to the two balloons 2, 1 can be configured with a flared outer surface ring or collar 4 (see FIG. 1) that prevents the balloon complex from being pushed in its entirety into the artificial or natural cavity in a patient 19 (see FIGS. 4 and 5). The ingress and egress port(s) can be incorporated into this flared surface 4 or can exit either or both of the balloons 2, 1 via separate tubing 11.

Temperature sensitive hydrogels undergo a solution (sol) to gel (gel) transition at a predetermined temperature. In the sol state, the hydrogel is free flowing. In the gel state, the gel sets up as a solid that does not flow and retains the shape of the vessel or container 10 in which it is contained. Such a material can be injected into the outer balloon 1 at room temperatures or at a cooled temperature as a free flowing liquid. Once it is inside the outer balloon 1, which can be at least partially or completely positioned inside a natural or artificial cavity in a patient 19, the hydrogel is exposed to physiologic temperatures (e.g., 96 to 104 degrees Fahrenheit). By using a hydrogel, whose sol-gel transition occurs at physiologic temperatures, the hydrogel can be used to fix the shape of the outer balloon 1 that is created by its contact with surrounding tissue 18. The time required for this to occur, and the time available to allow the outer balloon 1 to conform to the surrounding tissues 18, can be controlled by infusing water at less than physiologic temperatures or the sol-gel transition temperature, into the artificial or natural cavity of the patient 19, thereby bathing the outer balloon 1 in the water and reducing the temperature to which it is exposed for some period of time. This approach—bathing the surrounding cavity in a fluid that is below the sol-gel transition temperature, also can be used to cause a gel-sol transition so that the hydrogel can be removed from the outer balloon 1. The gel-sol transition can be aided by cycling cold water through the inner balloon 2, which will cool the hydrogel from the “inside out.”

An alternative approach is to use a hydrogel that has a sol-gel transition temperature that is greater than physiologic temperatures. Such a temperature can be achieved in the hydrogel by infusing water that is above physiologic temperature into the cavity surrounding the balloon or into the inner balloon 2. In this case, the gel-sol transition would occur naturally as the bathing fluid either cools down or is removed, and can be aided by circulating cold water through the inner balloon 2.

A higher than physiologic temperature also can be achieved in the hydrogel by turning on the ultrasound probe 5 and using its energy to warm the hydrogel as the probe 5 passes through the hydrogel. Since some portion of the ultrasound energy will be absorbed by the hydrogel during the treatment, this approach insures that the hydrogel will remain in a gel state for the entire duration of the treatment.

The hydrogel can be impregnated with various biologic substances selected to act in concert with the ultrasound, distinct from the ultrasound, or to be activated by the ultrasound. The material used for the membrane of the outer balloon 1 can be porous, thereby allowing substances suspended in the hydrogel to leach out of the hydrogel across the membrane and into the tissue against which the membrane is juxtaposed. The membrane used for the outer balloon 1 also can be nonporous. The molecular configuration of the hydrogel also can be used to modify the focal point of the therapeutic ultrasound probe. By using a gel with a diffraction index different from water, the gel can act as a lens that will change the focus of the ultrasound energy.

In one embodiment, fluid that has the density of water is transparent to ultrasound energy. Thus, depending on the actual tissue path length the beam travels through on its way to the geometric focal point of the crystal being used, the amount of energy attenuation by the tissue will vary, requiring that the power applied to the crystal may have to be varied in order to ensure that a constant amount of heat is delivered to the tissue at the focal point. If hydrogel is used that has a density in its gel state that is equivalent to that of tissue, the effective tissue path from the crystal to the focal point will become constant and independent of the actual tissue path length through which the beam travels, meaning that a constant power setting can be used to deliver the treatment, reducing the complexity of the physician interaction with the device.

Referring to FIGS. 4 and 5, in use, the dual balloon, with air having been evacuated from both balloons 2, 1 and their associated valves or ports closed in order to make the overall profile as small as possible, can be inserted into a natural or man-made orifice in a patient 19 with the collar 4 sitting on the orifice surface. A stylet can be inserted into the inner balloon 2 through the port to aid in the insertion of the balloons 2, 1 or the ultrasound probe 5 can be inserted in the inner balloon 2 prior to balloon insertion to stiffen the balloons. Once in place, and if the probe 5 was not used to insert the balloon(s), the probe 5 can be passed through the valve or port into the inner balloon 2. Water or another non-gaseous fluid can be circulated through the probe 5, thereby filling the inner balloon 2 with fluid 15 (see FIG. 5). The amount of non-gaseous fluid can be controlled by mechanisms internal or external to the probe control system. This water or non-gaseous fluid can be chilled in order to reduce heat buildup at the surface of the ultrasound crystal within the transducer or to produce a gel-sol transition at the end of the treatment.

In one embodiment, both balloons are filled with water, liquid and/or gel that has an acoustic velocity equal to, or very similar to water or tissue. This is to prevent beam reflections, focusing, or de-focusing at the interfaces, especially in instances where the user is not able to control or predict the shape of the balloons during use.

The balloon and probe 5 can be adjusted to be in the correct position for treating the targeted tissue based on the use of either on-board probe imaging capability or other means. The outer balloon 1 can then be filled with enough of the thermosensitive hydrogel to cause the outer balloon 1 to expand and engulf, surround or achieve significant contact with the region of tissue containing the targeted tissue 17. While the hydrogel is still in a free flowing state, fluid can be added to the inner balloon 2 to create the proper delivery depth, if required. Increasing the volume or size of at least the inner balloon 2 pushes the probe 5 further from the surface of the tissue being treated, thereby bringing the focal point of the probe 5 closer to the surface, while decreasing the volume or size of at least the inner balloon 2 brings the probe 5 closer to the surface tissue, thereby pushing the focal point of the probe 5 deeper into the tissue. Tissue depth can also be adjusted by controlling the amount of solution injected into the outer balloon.

The hydrogel can then be allowed, via physiologic means, or forced, by turning on the ultrasound probe 5 or instilling fluid above physiologic temperatures into the cavity, to undergo a sol-gel transition 16 (see FIG. 5). This causes the shape of the outer balloon 1 to become fixed, thereby immobilizing the surrounding tissue 18 relative to the outer balloon 2 and the outer balloon 2 relative to the inner balloon 1. The ultrasound treatment can then be delivered, taking advantage of the heat transferred to the hydrogel to maintain it in the gel state.

Once the ultrasound delivery is completed, the hydrogel can be forced to undergo a gel-sol transition by infusing cold water into the treatment cavity, by infusing cold water into the inner balloon 2, or by letting the gel cool to physiologic temperatures if a hydrogel was used that has a sol-gel transition temperature greater than body temperature. As the gel-sol transition occurs, the hydrogel can be removed from the outer balloon 1 by applying suction to the egress port. Once all or substantially all of the hydrogel is removed from the outer balloon 1, the probe 5 can be removed from the inner balloon 2, the water removed from the inner balloon 2, suction applied to both balloons 2, 1 to reduce their profile as much as possible if needed, and the entire balloon complex removed from the patient. It is understood by those skilled in the art that one or more of the above-described steps can be accomplished in an alternative or different order.

In one embodiment, for high intensity focused ultrasound (HIFU) to work as intended, there needs to be a fluid space surrounding the transducer(s) that is free-flowing so that the probe can be moved around inside that space (e.g., the body orifice), which is potentially a relatively large volume. In order to be able to introduce the probe into a confined environment (e.g., the body orifice) whose access is smaller than the end size of the volume, in one embodiment the fluid needs to be able to be injected into the space surrounding the transducer(s) after the probe is inserted.

In addition, while it is desirable that the probe be able to be moved around in order to position it properly within the body orifice, once the probe is in place, it is desirable that the probe be immobilized, or the energy from the probe will not be concentrated at the correct location. In one embodiment, this means that the probe has a moveable fluid space surrounding it to be used, but a rigid means surrounding the fluid space to hold the “fluid container” in place. A way to accomplish this is to have a material that is fluid during introduction, but that can be made rigid after introduction. A hydrogel meets this requirement, where it is not being used to occupy and maintain a defined space, but is being used to mold to an existing space and then to hold that space rigid.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I/we claim:

1. A system for delivering ultrasound therapy, the system comprising:
a focused ultrasound probe including a transducer; and
a balloon complex including a first balloon and a second balloon, the first balloon being located inside the second balloon and surrounding the transducer, the first balloon containing a first fluid, the first fluid being a non-gaseous fluid, the second balloon containing a second fluid, the second fluid being thermosensitive hydrogel, the thermosensitive hydrogel transforming from a fluid at room temperature to a gel at a higher temperature, the first and second balloons creating a fluid interface between the probe and targeted tissue of a patient.

2. The system according to claim 1, wherein the thermosensitive hydrogel immobilizes the first balloon relative to the second balloon and the second balloon relative to surrounding tissue of the patient.

3. The system according to claim 1, wherein the first balloon acts as a fluid interface, a mechanism for cooling, and a mechanism for changing tissue depth of a focal point of the probe.

4. The system according to claim 1, wherein at least a portion of the probe is positioned within the first balloon, and wherein the first and second balloons stabilize the probe with respect to the targeted tissue.

5. The system according to claim 1, wherein the transducer includes at least one therapy crystal, the probe delivering thermal energy to the targeted tissue.

6. The system according to claim 5, wherein the transducer includes at least one imaging crystal.

7. The system according to claim 1, further comprising at least one port to allow air to be removed from the second balloon and inject and remove fluid from the second balloon, the at least one port also allowing the probe to be removed from the first balloon and pass at least a portion of the probe into the first balloon.

8. The system according to claim 1, wherein a neck of the balloon complex includes a collar with a flared outer surface, the flared outer surface preventing the balloon complex from being pushed entirely into an orifice of the patient.

9. The system according to claim 1, wherein the first fluid is different than the second fluid.

10. A system for delivering ultrasound therapy, the system comprising:
a focused ultrasound probe including a transducer;
a balloon complex including a first balloon and a second balloon, the first balloon surrounding the transducer and located at least partially inside the second balloon, the first balloon being configured to receive a first fluid, the second balloon being configured to receive a second fluid, the second fluid being different from the first fluid, the first balloon being configured to expand to form an elliptical or circular shape having a convex exterior surface without any concave portions; and
at least one port including tubing extending from an interior of the second balloon to an exterior of the second balloon, the at least one port allowing (i) air to be removed from the second balloon and (ii) non-gaseous fluid to be injected into or removed from the second balloon.

11. The system according to claim 10, wherein the first fluid is a non-gaseous fluid, the second fluid being a thermosensitive hydrogel configured to transform from a fluid at room temperature to a gel at a predetermined higher temperature, and the thermosensitive hydrogel being configured to immobilize (i) the first balloon relative to the second balloon and (ii) the second balloon relative to a patient, the first and second balloons being configured to create a fluid interface between the probe and targeted tissue of the patient.

12. The system according to claim 10, wherein the first balloon is configured to act as a fluid interface, a mechanism for cooling, and a mechanism for changing tissue depth of a focal point of the probe.

13. The system according to claim 10, wherein the probe includes at least one therapy crystal and at least one imaging crystal, the probe delivering thermal energy to the targeted tissue.

14. The system according to claim 10, wherein a neck of the balloon complex includes a collar with a flared outer surface, the flared outer surface being configured to prevent the balloon complex from being pushed entirely into an orifice of the patent.

15. A system for delivering ultrasound therapy, the system comprising:
a focused ultrasound probe including a transducer, the transducer including at least one therapy crystal configured to deliver thermal energy to targeted tissue of a patient;
a balloon complex including a first balloon, a second balloon and a neck, the first balloon being located at least partially inside the second balloon and surrounding the transducer, the neck being located outside of the first and second balloons, the first balloon containing a first fluid, the first fluid being a non-gaseous fluid, the second balloon containing a second fluid, the second fluid being a thermosensitive hydrogel configured to transform from a fluid at room temperature to a gel at a predetermined higher temperature, the second fluid being different from the first fluid, the first balloon being expandable to form an elliptical or circular shape having a convex exterior surface without any concave portions, the first and second balloons creating a fluid interface between the probe and targeted tissue of the patient, the second fluid immobilizing (i) the first balloon relative to the second balloon and (ii) the second balloon relative to the patient, the neck including a collar with a flared outer surface configured to prevent the balloon complex from being pushed entirely into an orifice of the patent; and
at least one port including tubing extending from an interior of the second balloon to an exterior of the second balloon, the at least one port allowing (i) air to be removed from the second balloon and (ii) non-gaseous fluid to be injected into or removed from the second balloon.

16. The system according to claim 15, wherein the transducer includes at least one imaging crystal.

17. The system according to claim 15, wherein the at least one port is configured to allow the probe to be removed from the first balloon and pass at least a portion of the probe into the first balloon.
